(19) United States
(12) Patent Application Publication
(10) Pub. No.: US 2026/0157713 A1
(43) Pub. Date: Jun. 11, 2026

(54) DRIVE ASSEMBLY FOR A 3D X-RAY IMAGING SYSTEM

(71) Applicant: 3DIO, Inc., Orem, UT (US)

(72) Inventors: Thomas L. YOUD, Holladay, UT (US); Kolby Black, Salem, UT (US); Brad Allen, Orem, UT (US)

(21) Appl. No.: 18/977,278

(22) Filed: Dec. 11, 2024

(51) Int. Cl. G01N 23/046

(52) U.S. Cl. CPC A61B 6/4476; A61B 6/51; A61B 6/56; A61B 6/027

(57) ABSTRACT

This application describes dental X-ray devices and systems that use a rotating X-ray source on one side of a patient to take two-dimensional (2D) images from multiple angles and then reconstruct a three-dimensional (3D) image using those 2D images. The X-ray imaging system contains a drive assembly comprising an X-ray source configured to rotate around a shaft where the X-ray source is attached to a support plate, a motor configured to rotate the support plate around a shaft using a belt and a gear, a slewing ring connected to the support plate which is configured to rotate the support plate, a slip ring attached to the shaft, and a sensor attached to a slotted encoder that rotates with the support plate and tracks the location of the X-ray source. With this drive assembly, the X-ray source of the 3D imaging system can rotate freely in a safe and reliable manner and be ready to capture an X-ray image nearly immediately on start-up. Other embodiments are described.

### FIELD

This application relates generally to X-ray equipment, including X-ray devices and X-ray systems. More specifically, this application relates to a drive assembly for dental X-ray devices and systems that use a rotating X-ray source on one side of a patient to take two-dimensional (2D) images from multiple angles and then reconstruct a three-dimensional (3D) image using those 2D images.

### BACKGROUND

X-ray imaging systems typically contain an X-ray source and an X-ray detector. X-rays (or other types of radiation used for imaging) are emitted from an X-ray tube in the X-ray source and impinge on the X-ray detector to provide an X-ray image of the object or objects that are placed between the X-ray source and the detector. The X-ray detector is often an image intensifier or even a flat panel digital detector.

Intra-oral radiography is a standard imaging technique in dentistry, with bite-wing and periapical X-rays considered the standard of care in dental practice. However, there are many features of the tooth anatomy that are not visible in standard intra-oral radiographs since they are only two-dimensional (2D) projections of a three-dimensional (3D) structure. Accordingly, 3D imaging is often used in some dental procedures. One form of 3D imaging, cone-beam computed tomography (CBCT), is becoming widely used in dental imaging. In CBCT, a patient's head is positioned between a large imaging detector and an opposing X-ray source. The detector and source rotate around the head while taking multiple 2D images. Using these 2D images, a 3D image of the patient's oral and maxillofacial anatomy can be reconstructed. This technique works very well for imaging the entire oral cavity and displaying the spatial relationships between the teeth and other bony structures located in the head of a patient. But it is often not used to image just a few teeth (or a single tooth) because of the increased radiation that the patient is subjected to.

### SUMMARY

This application relates generally to X-ray equipment, including X-ray devices and X-ray systems. More specifically, this application describes dental X-ray devices and systems that use a rotating X-ray source on one side of a patient to take two-dimensional (2D) images from multiple angles and then reconstruct a three-dimensional (3D) image using those 2D images. The X-ray imaging system contains a drive assembly comprising an X-ray source configured to rotate around a shaft where the X-ray source is attached to a support, a motor configured to rotate the support around a shaft using a drive linkage and a drive member, a drive bearing connected to the support which is configured to rotate the support, a slip ring attached to the shaft, and a sensor attached to an encoder that rotates with the support and tracks the rotation of the X-ray source. With this drive assembly, the X-ray source of the 3D imaging system can rotate freely in a safe and reliable manner and can be ready to capture an X-ray image nearly immediately on start-up.

### BRIEF DESCRIPTION OF THE DRAWINGS

The following description can be better understood in light of the Figures which show various embodiments and configurations of the X-ray tubes and X-ray devices in which they are used.

FIG. 1 shows a view of some embodiments of a 3D X-ray imaging system.

FIG. 2 shows a view of some embodiments of an X-ray head of a 3D X-ray imaging system.

FIGS. 3-4 show some embodiments of the components contained in the housing of an X-ray head of a 3D X-ray imaging system.

FIG. 5 shows some embodiments of a 3D X-ray imaging system mounted to equipment in a dental office.

FIGS. 6-7 show some embodiments of a drive assembly for 3D X-ray imaging systems.

Together with the following description, the Figures demonstrate and explain the principles of the structures and methods described herein. In the drawings, the thickness and size of components may be exaggerated or otherwise modified for clarity. The same reference numerals in different drawings represent the same element, and thus their descriptions will not be repeated. Furthermore, well-known structures, materials, or operations are not shown or described in detail to avoid obscuring aspects of the described devices.

### DETAILED DESCRIPTION

The following description supplies specific details in order to provide a thorough understanding. Nevertheless, the skilled artisan will understand that the described X-ray systems can be implemented and used without employing these specific details. Indeed, the described systems and methods can be placed into practice by modifying the described systems and methods and can be used in conjunction with any other apparatus and techniques conventionally used in the industry. For example, while the description below focuses on X-ray devices and systems that can be used in imaging systems for dental imaging, they can be used for other purposes such as medical imaging, veterinary imaging, industrial inspection applications, and anywhere where X-ray radiography equipment is currently being used to generate a standard 2D X-ray image.

In addition, as the terms on, disposed on, attached to, connected to, or coupled to, etc. are used herein, one object (e.g., a material, element, structure, member, etc.) can be on, disposed on, attached to, connected to, or coupled to another object—regardless of whether the one object is directly on, attached, connected, or coupled to the other object or whether there are one or more intervening objects between the one object and the other object. Also, directions (e.g., on top of, below, above, top, bottom, side, up, down, under, over, upper, lower, lateral, orbital, horizontal, etc.), if provided, are relative and provided solely by way of example and for ease of illustration and discussion and not by way of limitation. Where reference is made to a list of elements (e.g., elements a, b, c), such reference is intended to include any one of the listed elements by itself, any combination of less than all of the listed elements, and/or a combination of all of the listed elements. Furthermore, as used herein, the terms a, an, and one may each be interchangeable with the terms at least one and one or more.

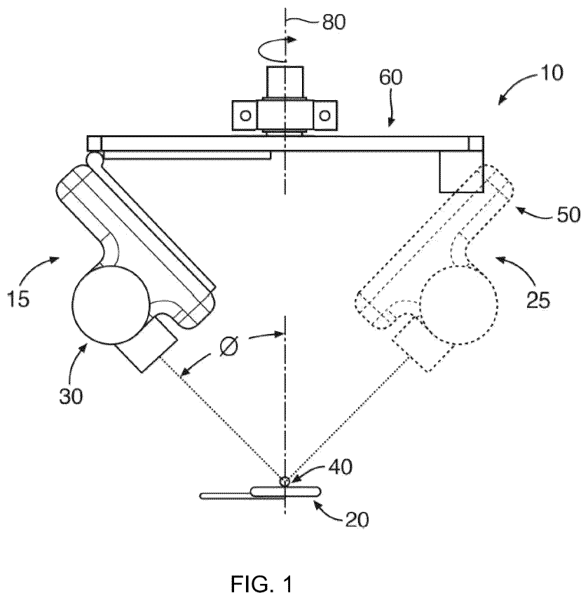

Some embodiments of the 3D X-ray imaging systems (or X-ray imaging system or X-ray system) are illustrated in FIG. 1. FIG. 1 shows the geometry of a 3D X-ray imaging system with a rotation scheme shown with the X-ray source at position 15 and position 25 around axis of rotation 80. In FIG. 1, the 3D imaging system 10 comprises an imaging detector 20 that is located inside the mouth (not shown). The imaging detector 20 can be substantially stationary adjacent to the tooth (or teeth) 40 of a patient, or even completely stationary relative to the tooth using any stabilizing mechanism such as a bite block or other sensor holder device. The 3D imaging system 10 also contains an X-ray source 30 that is optionally located within a housing (not shown) that can be connected to a support arm 60.

The 3D X-ray imaging system 10 can contain any X-ray source 30 and X-ray detector 20 that allows the system 10 to take multiple 2D X-ray images or radiographs. The X-ray source 30 can contain any source that generates and emits X-rays, including a standard stationary anode X-ray source, micro-focus X-ray source, rotating anode X-ray source, and/or a carbon nanotube or micro-machined (Spindt cathode) X-ray source. In some embodiments, the X-ray source can operate with about 40 kV to about 90 kV and from about 1 mA to about 10 mA. In other embodiments, the X-ray source can operate with about 55 kV to about 75 kV and between about 3 mA and about 9 mA. In still other embodiments, the X-ray source can operate with about 60 kV to about 70 kV and between about 4 mA and about 7 mA. In some embodiments, the X-ray source and X-ray detector can be made modular so that different sizes and types of X-ray sources and X-ray detectors can be used.

The X-ray detector 20 can contain any detector (or sensor) that detects X-rays, including an image intensifier, CCD array, CMOS/scintillator array, and/or a digital flat panel detector. In some configurations, the detector can have a substantially square shape with a length on one side ranging from about 2 cm to about 6 cm. In other configurations, though, the X-ray detector 20 does not need to have a substantially square shape but can have a rectangular shape to roughly match the size of a tooth of a patient. In yet other embodiments, though, the X-ray detector 20 does not need to have a substantially square or rectangular shape.

In some configurations, the X-ray detector 20 can be synchronized and/or aligned with the X-ray source 30 so that the X-ray system 10 can take multiple images with high efficiency. This synchronization can be performed by controlling both the X-ray detector and the X-ray source using an internal or external controller, such as a computer, or by configuring the detector to collect data when it first detects X-rays, thereby only requiring control of, or timing, of the X-ray source so it emits the X-ray pulses when desired.

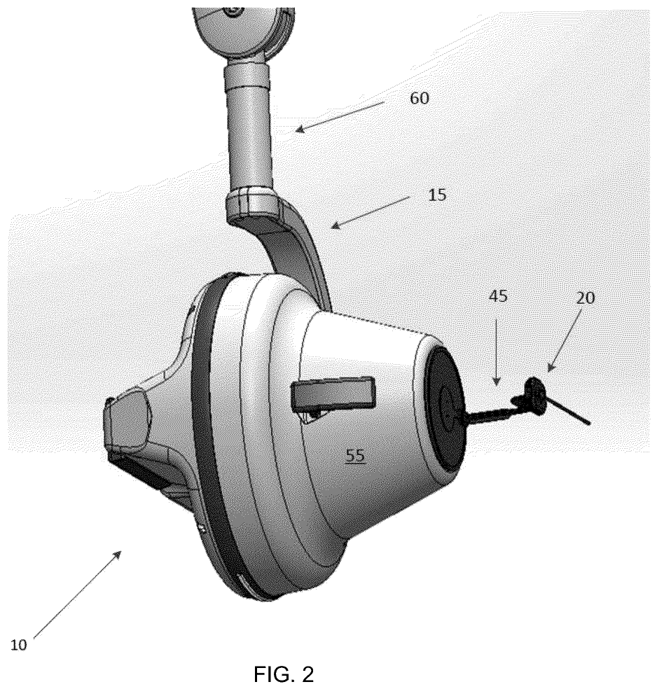

Other embodiments of the 3D X-ray imaging system are shown in FIG. 2. The 3D X-ray imaging system 10 can contain an X-ray head 55 that contains an X-ray source 30 (not shown) and high voltage electronics (not shown) on the inside of the head 55. The high voltage electronics can provide typically between about 40,000 volts and about 200,000 volts for most medical applications, and/or about 50,000 volts up to about 70,000 volts for most dental applications, whether DC or AC current. In other embodiments, the high voltage electronics can provide any combination of these voltage amounts. The X-ray imaging system also contains an arm 60 that is coupled to the X-ray head 55. The X-ray head 55 can be configured to be removably connected to the arm 60 using a yoke 15 so that when the X-ray head 55 is disconnected, it can be removed from the remainder of the X-ray system 10.

The X-ray head 55 can also be attached to an X-ray detector 20. The X-ray detector 20 can contain any detector (or sensor) that detects X-rays, including an image intensifier, CMOS camera, and/or a digital flat panel detector. The X-ray detector 20 can be connected to the X-ray head 55 using an aligner 45 that helps keep the X-ray detector 20 properly positioned with respect to the tooth of a patient and the X-ray source while an image is taken.

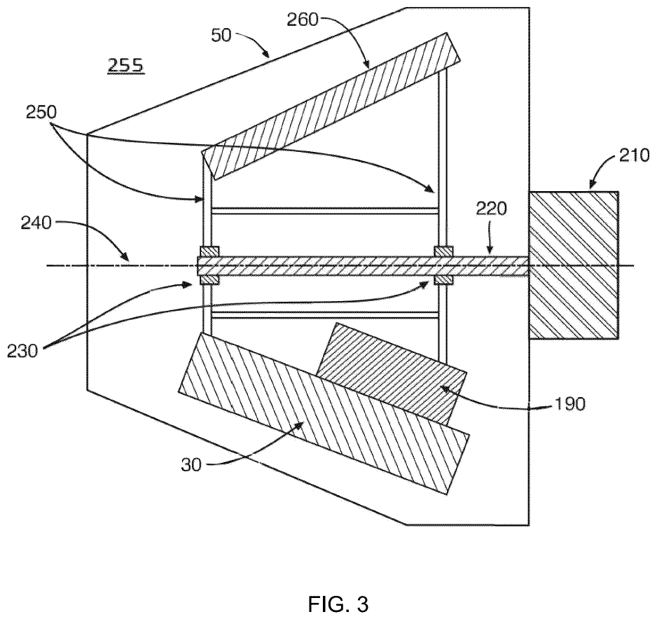

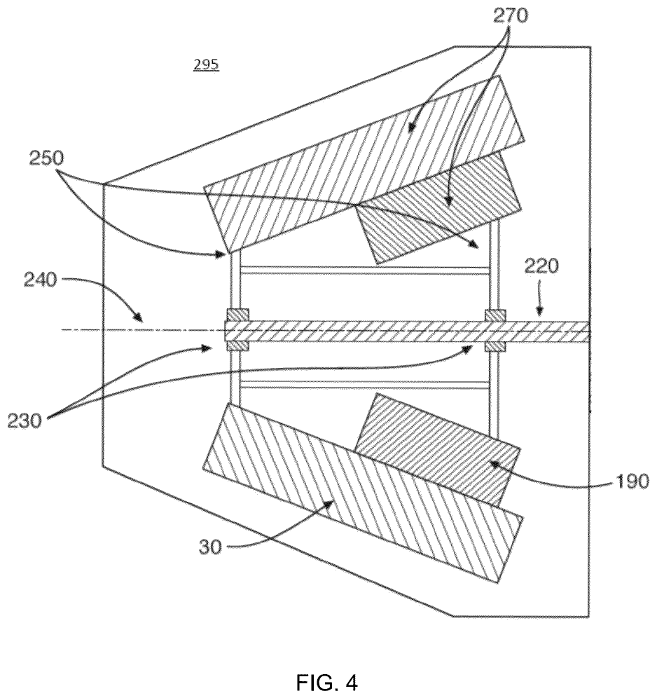

Other embodiments of the X-ray head are shown in FIGS. 3-4. As shown in detail in FIG. 3, the X-ray source 30 can be contained in housing 50 of the X-ray head 255. The housing 50 can be configured with a first portion enclosing the X-ray source 30 as shown in FIG. 3. The housing 50 also encloses a second portion that contains a counterweight 260 for the X-ray source 30, power electronics 190, and other components, which facilitates smooth vibration-free rotary motion of the source 30. The X-ray source 30 and its associated power electronics 190 and the counterweight 260 are located as necessary on rotating mechanical assembly 250 which supports the X-ray source 30, the power electronics 190, counterweight 260, and other components (not shown) to properly balance the rotating mechanical assembly 250. The rotating mechanical assembly 250 is mounted to axle 220 (or other mechanical device to support the mechanical assembly) with an axis of rotation 240 using the bearings and/or electric motor assembly 230 to drive rotation of the mechanical assemble 250.

As shown in FIG. 3, the housing 50 can also be configured so that it is a single part that encloses both the X-ray source 30 and these components. In other configurations, the housing can be separated into different parts or portions to contain the X-ray source 30 and other components. As shown in FIG. 3, the electronic components for control and power conditioning 210 can be located just outside of the housing 50. In other embodiments, these electronic components 210 can be located on the support arm or other convenient location. In yet other embodiments, these electronic components 210 can be located internal to the housing 50.

Indeed, as shown in the embodiments in FIG. 4, these electronic components 210 have been moved from the outside of the X-ray head 295 so that they are located internal to the housing of the X-ray head 295. The electronic components can be configured so that they rotate within the X-ray head 295 along with the X-ray source 30. In the embodiments shown in FIG. 4, these internal electronic components can include both a power supply and a power source (collectively labeled as 270). In these configurations, these internal electronic components can operate as a counterweight to the X-ray source 30, thus eliminating the separate counterweight that is shown in FIG. 3. In other configurations, the internal electronic components can be configured as a separate power source and power supply that, along with the X-ray source, all counterbalance each other. In yet other configurations, the X-ray source can be counter balanced by the power supply and the power source is located near the motor or contained in the stand. In even other configurations, the power supply and the X-ray source could be combined and counterbalanced by the power source, or they could be separate (and counter balancing each other), with the power source either combined with the power supply or moved to an area outside of the rotation. In other words, there could be combinations of these three parts and any of them, except the X-ray source, could be placed outside of the rotation.

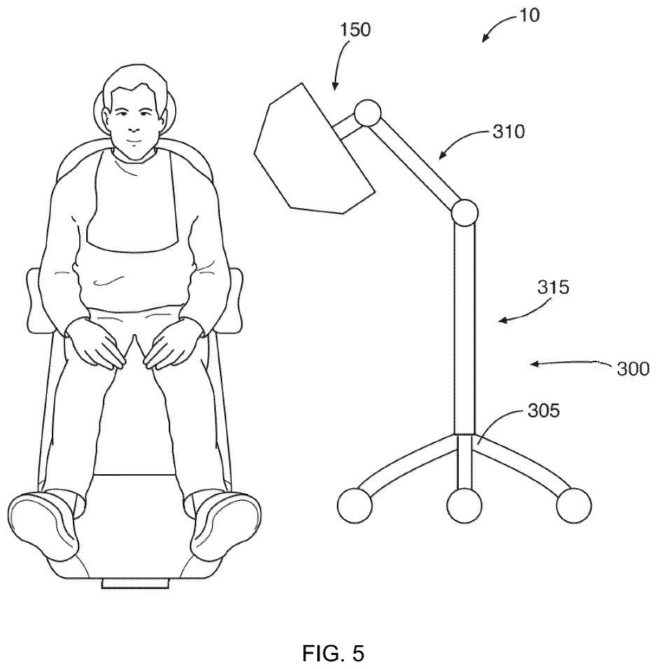

In other configurations, the 3D X-ray imaging systems can contain a removable power source (such as a battery) and a power supply. In these configurations, the power source and/or the power supply can be located on or in any supporting structure which the 3D imaging systems might be used with. For example, the supporting electronics for the power source and the power supply, as well as the supporting electronics for the image display and for the wireless data upload described herein, can also be located internal or external to a support structure to which the housing 50 is connected, such as stand 300 shown in FIG. 5. Thus, in these configurations, the X-ray imaging system 10 does not require an external power cord. Incorporating the power source (i.e., the battery), the power supply, and the supporting electronics all in or on the external structure allows the 3D imaging systems to be portable and moved from one dental station to another. With such a configuration, the power source can easily be replaced or swapped. Of course, if needed, the X-ray imaging system 10 can be configured so that it is alternately, or additionally, powered using external power from a power cord that is plugged into a wall outlet. In other configurations, multiple power supplies can be provided for the source, detector, and control electronics.

The support arm 60 can have any configuration that allows the X-ray source 30 in the housing to direct X-ray beams at the desired angle through the tooth (or teeth) and on the detector 20. In the embodiments shown in FIG. 1, the support arm 60 has a substantially straight configuration with the X-ray head connected to an end thereof. In other configurations, the support arm need not be straight and can have jointed or articulated sections such as those shown in FIG. 2. In yet other configurations, the X-ray head can be connected to the support arm 60 at any location other than its end.

In some embodiments the 3D X-ray imaging systems can be attached to and used with an external support structure, as illustrated in FIG. 5. In this Figure, the 3D imaging system 10 with a frame 150 can be connected to a stand 300. The stand 300 contains a base 305 and an arm 315 extending upwards towards an extension 310. The extension 310 is connected to the joint which is, in turn, connected to the frame 150 of the 3D imaging system 10. In other configurations, the 3D imaging system 10 can be connected to a movable support structure. In such configurations, the movable support structure can be configured to move across a floor while supporting the 3D imaging system 10. Thus, the movable support structure can comprise one or more wheels, shelves, handles, monitors, computers, stabilizing members, limbs, legs, struts, cables, and/or weights (to prevent the weight of the imaging arm and/or any other component from tipping the movable support structure). Thus, the movable support structure could comprise a wheeled structure connected to a stand that contains the joint that is connected to the frame 150 of the 3D imaging system 10.

The volume and weight of the 3D imaging system should be minimized as much as possible for ease of use and ease of alignment. To reduce the size and/or weight, the 3D imaging system can be equipped with small and light-weight components. Over the last decade, there have been significant innovations in miniaturization of X-ray tubes. These lightweight sources can greatly simplify the task of motion automation for the 3D imaging systems described herein. In addition, newer CMOS detectors are much more sensitive, resulting in less dose to the patient than required with conventional CCD designs. The new CMOS detectors can also have very high read-out speeds allowing for rapid collection and transmission of multiple 2D images. To achieve the 3D imaging systems described herein, the X-ray source should fit within a volume of about 13 cm×about 7 cm×about 8 cm and weigh less than about 1.9 Kg. As well, CMOS detectors with capability of at least 5 (or more) frames per second should be used. One way to achieve X-ray sources that meet these requirements would be to use a carbon-nanotube or Spindt-cathode (micro-machined silicon or similar technology) electron source within the X-ray source 30.

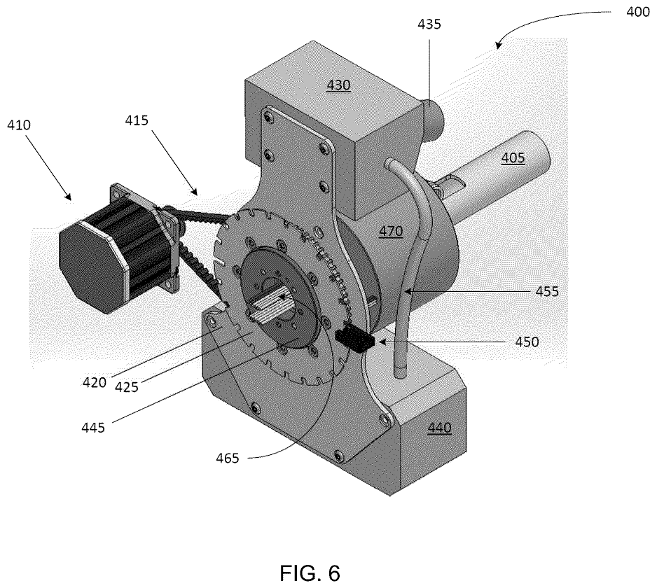

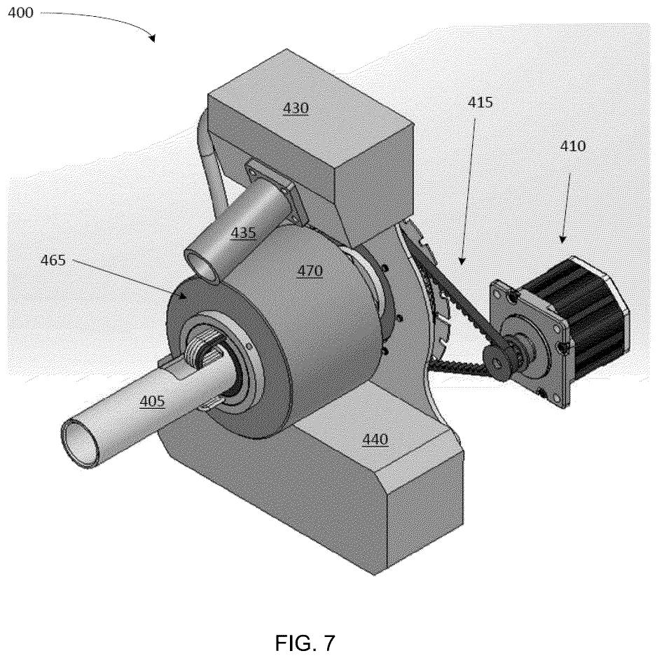

Because the X-ray source rotates within the X-ray head, in some embodiments the X-ray head can be configured with components that assist with—and not detract from—this rotation. In some configurations, the X-ray head of the 3D X-ray imaging systems described herein can be configured with the drive assembly (or drive system) 400 illustrated in FIGS. 6-7. In these configurations, the drive assembly 400 can contain a motor 410 that is used to rotate the X-ray source 430. The motor 410 can be connected to a support in the shape of an elongated plate (e.g., support plate 420) with two portions that is configured to rotate via a drive linkage that is connected to a drive member that is attached to the support plate 420. In some configurations, the drive linkage can comprise belt 415 and the drive member can comprise gears.

The X-ray system 400 also contains a collimator 435 located proximate the X-ray source 430 and is used to control the direction of the X-ray beam emitted from the X-ray source. The X-ray source 430 can be mounted to a first portion of support plate 420. The electronics 440 supporting the operation of the X-ray source 430 (i.e., the supporting electronics) can be mounted to a second portion of the support plate 420 that is roughly opposite, or across the axle from, the first portion where the X-ray source 430 is mounted. When the motor is activated, the drive linkage (e.g., belt 415) turns the drive members (e.g., gears) which, in turn, rotate support plate 420 along with the X-ray source 430 and the supporting electronics 440 that are mounted, respectively, to first and second portions of the support plate 420.

The X-ray source 430 can also be electrically connected to the supporting electronics 440 using cable 455. In some configurations, the cable 455 can contain both high voltage and low voltage wires. In other configurations, the cable 455 can comprise the high voltage wires running from the X-ray power supply in the supporting electronics 440 to the tube of the X-ray source 430 while another cable (not shown) can comprise the low voltage wires running from the X-ray power supply to the X-ray source 430.

The X-ray source 430, supporting electronics 440, and support plate 420 can rotate around an axis that is located roughly central to shaft 405 as shown in FIG. 6. As shown in the embodiments of FIG. 7, shaft 405 can contain a hollow center through which cables and/or wires (not shown) can run through the drive system 400. These cables and/or wires can electrically connect the X-ray detector to the rest of the X-ray system.

The drive assembly 400 also contains a drive bearing. In some configurations, the drive bearing comprises slewing ring 445. The slewing ring 445 (or slewing bearing) is a rotational rolling-element bearing that supports the rotation of the support plate 420. The slewing ring 445 can comprise ball bearings, plastic bearings, and/or other bearing surfaces. Compared to normal ball bearings, the rings in the slewing ring 445 are wider. The slewing ring 445 contains holes drilled in it so that it can be attached to the support structure 420. Seals can be provided between the rings of the slewing ring 445 to protect the rolling elements. The drive bearing (e.g., slewing ring 445) can be used to rotate the X-ray source 430 in either direction (clockwise or counter-clockwise). When used with the drive bearing (e.g., slewing ring 445), the drive linkage (e.g., belt 415) allows for the use of gear ratios to get better torque and control from the motor 410 rather than the use of a direct drive. This configuration helps to lower costs of the X-ray imaging system. The use of a belt rather than a direct drive or a geared drive also provides mechanical isolation between the motor and the rotating elements that bear the X-ray source 430 for better vibration control.

The drive assembly 400 also contains a slip ring 470 that is located around shaft 405. A slip ring is a type of rotational electromechanical device (or rotary electrical connection) that passes electrical power and/or electronic signals across a rotational mechanical connection between a stationary and a rotating structure. The slip ring can rotate in one direction for an indefinite number of revolutions, helping to simplify the design of the drive system 400. In some embodiments, the slip ring 470 contains a stationary graphite/metal contact (or brush) which rubs on the outside diameter of a rotating metal ring. As the metal ring turns, the electric current and/or signal can be conducted through the stationary brush to the metal ring to make the connection. Additional ring/brush assemblies can be stacked along the shaft 405 if more than a single electrical circuit is needed. The slip ring 470 allows the electronic signals and/or power to be passed across the rotational joint regardless of the number of rotations and/or direction of rotation of the X-ray source 430. This configuration both simplifies the control of the rotation, as well as lowers the risk of damage/disconnect of the electrical signals. Thus, the slip ring 470 allows rotation of the supporting electronics 440 without worrying about any cables and/or wires (such as wires 455) getting tangled or needing to be unwound. In some configurations, the slip ring 470 can be used to allow continual power from the power source (that is not rotating) to the power supply.

In other configurations, properly-designed wires or cables could be used to pass this electrical power or electronic signals across the rotational mechanical connection. A downside to using wires or cables is that after a few turns, the wires can become twisted or wrapped up to the point that the wires and cables may be broken and/or the rotational motion will be impeded by the tension that develops in the wires or cables. This downside can be compensated by rotating the structure in the opposite direction in order to “unwind” the wires or cables. In these configurations, the X-ray imaging system can be operated to alternate the direction of rotation from one image acquisition to another. There is little to no preference for one direction of rotation over the other since the support plate and X-ray source only revolve about 1½ to 2 times per image acquisition in these configurations.

The wires 465 in the drive assembly 400 are stationary wires that come from the back of the X-ray head and deliver power and/or signal connections to the slip ring 470. These wires 465 can deliver any needed power and/or signals from the rest of the X-ray imaging system to the X-ray head. The stationary wires are the stator of the slip ring 470 where the X-ray source 430 is physically and electrically connected to the rotor portion of the slip ring 470.

The drive assembly 400 also contains an encoder 425, as shown in FIG. 6. The encoder is a slotted disk connected to the rotating support plate 420 and/or the slewing ring 445. In some configurations, the encoder 425 can contain any number of slots that match the number of X-ray image frames taken in a desired rotation which, in some embodiments, is a rotation of about 360 degrees. In other embodiments, the desired rotation might be 720 degrees, or 270 degrees, or perhaps 180 degrees. In some configurations, encoder 425 can contain between about 24 to about 36 slots, while in other configurations the number of slots might be between 18 and 42 slots, or perhaps between 48 and 72 slots.

One of those slots can be designated as the primary slot. It can be configured differently than the rest, such as by being deeper or wider. A sensor 450 (or sensors), such as any infrared sensor, can be used to read out the positions of slots of the encoder 425 using the primary slot as a reference point. Thus, the encoder 425 can be used for timing the firing of the X-ray source 430, the detector image capture, as well as any speed and position feedback for the drive assembly 400.

In the X-ray imaging system, the X-ray source 430 rotates around the shaft 405 and is driven by motor 410. This configuration, however, can complicate the operation of the X-ray system since it makes it difficult to pass power and signals over the rotating joint. This complication can be overcome by using the slip ring 470 of the drive assembly 400.

These 3D X-ray imaging systems with the described drive assembly exhibit several valuable features. One such feature is that the X-ray source can rotate in a safe and reliable manner. Another such feature is that slip ring 470 provides means for the system to rotate freely in either direction. Thus, the system does not need to unwind any cabling after completing a sequence nor does it need to start or end at a specific rotation position. As well, since the X-ray source can rotate in both directions, the X-ray system can take multiple images in quick succession. This configuration also allows the system to rotate freely during transportation and installation so the system can be ready for operation at any time. The encoder disk 425 also synchronizes the motor speed with the X-ray source 430, allowing for a reliable system even with variations in motor function.

Another helpful feature of the drive systems described herein is that the 3D X-ray imaging system has full control and flexibility of operating parameters. These operating parameters include knowing the location of the X-ray source, being able to control or vary the rotational speed at any time, as well as start, pause, or change rotational direction all while maintaining constant electrical contact across the rotating joint.

Another important feature of the drive systems described herein is that the encoder disk allows for the X-ray source to begin operating and taking X-ray images at any position. This functionality shortens the capture time of the images by not requiring the system to move the X-ray source into position before beginning a capture of an X-ray image. It also simplifies the functions of the system between image captures as it is not trying to find or hold a start position.

In addition to any previously indicated modification, numerous other variations and alternative arrangements may be devised by those skilled in the art without departing from the spirit and scope of this description, and appended claims are intended to cover such modifications and arrangements. Thus, while the information has been described above with particularity and detail in connection with what is presently deemed to be the most practical and preferred aspects, it will be apparent to those of ordinary skill in the art that numerous modifications, including, but not limited to, form, function, manner of operation and use may be made without departing from the principles and concepts set forth herein. Also, as used herein, the examples and embodiments, in all respects, are meant to be illustrative only and should not be construed to be limiting in any manner.

1. A drive assembly for a 3D X-ray imaging system, comprising:
a support configured to be attached to an X-ray source;
a motor configured to rotate the support around a shaft using a drive linkage and drive member;
a drive bearing connected to the support and configured to rotate with the support;
a slip ring attached to the shaft; and
a sensor attached to an encoder that rotates with the support and tracks the location of the X-ray source.

2. The drive assembly of claim 1, wherein the X-ray source is attached to a first portion of the support and electronics supporting the operation of the X-ray source are attached to a second, opposing portion of the support.

3. The drive assembly of claim 2, wherein the X-ray source and the supporting electronics act as counterweights to each other as the support rotates around the shaft.

4. The drive assembly of claim 2, wherein the X-ray source and supporting electronics are connected by high voltage and/or low voltage wires.

5. The drive assembly of claim 1, wherein the support comprises a plate that can rotate in a clockwise or counter-clockwise direction.

6. The drive assembly of claim 1, wherein the drive bearing contains a slewing ring that comprises ball bearings, plastic bearings, and/or other bearing surfaces.

7. The drive assembly of claim 1, wherein the slip ring conducts an electrical current and/or a signal without using wires.

8. The drive assembly of claim 1, wherein the encoder comprises a plurality of slots as a function of the number of X-ray image frames to be taken while the X-ray source rotates around the shaft.

9. The drive assembly of claim 8, wherein the encoder further comprises a primary slot configured to be used as a reference when activating the X-ray source and capturing an image using an X-ray detector.

10. An X-ray imaging system, comprising:
an X-ray source;
an X-ray detector;
a support attached to the X-ray source;
a motor configured to rotate the support around a shaft using a drive linkage and drive member attached;
a drive bearing connected to the support and configured to rotate with the support;
a slip ring attached to the shaft; and
a sensor attached to an encoder that rotates with the support and tracks the location of the X-ray source.

11. The X-ray imaging system of claim 10, wherein the X-ray source is attached to a first portion of the support and electronics supporting the operation of the X-ray source are attached to a second, opposing portion of the support.

12. The X-ray imaging system of claim 11, wherein the X-ray source and the supporting electronics act as counterweights to each other as the support rotates around the shaft.

13. The X-ray imaging system of claim 11, wherein the X-ray source and supporting electronics are connected by high voltage and/or low voltage wires.

14. The X-ray imaging system of claim 10, wherein the support comprises a plate that can rotate in a clockwise or counter-clockwise direction.

15. The X-ray imaging system of claim 10, wherein the drive bearing contains a slewing ring that comprises ball bearings, plastic bearings, and/or other bearing surfaces.

16. The X-ray imaging system of claim 10, wherein the slip ring conducts an electrical current and/or a signal without using wires.

17. The X-ray imaging system of claim 10, wherein the encoder comprises a plurality of slots as a function of the number of X-ray image frames to be taken while the X-ray source rotates around the shaft.

18. The X-ray imaging system of claim 17, wherein the encoder further comprises a primary slot configured to be used as a reference when activating the X-ray source and capturing an image using the detector.

19. An imaging method, comprising:
providing an X-ray imaging system on a first side of an object to be imaged, the imaging system comprising:
an X-ray source;
a support attached to the X-ray source;
a motor configured to rotate the support around a shaft using a drive linkage and drive member attached to the support;
a drive bearing connected to the support and configured to rotate with the support;
a slip ring attached to the shaft; and
a sensor attached to an encoder that rotates with the support and tracks the location of the X-ray source;
positioning a substantially stationary X-ray detector on an opposite side of the object;
rotating the X-ray source around the shaft to multiple positions;
collecting multiple two dimensional (2D) images of the object when the X-ray source is located in the multiple positions; and
reconstructing a three-dimensional (3D) image using the multiple 2D images.

20. The method of claim 19, wherein the object comprises a tooth.
